(19) United States
(12) Patent Application Publication
(10) Pub. No.: US 2022/0008161 A1
(43) Pub. Date: Jan. 13, 2022

(54) INFORMATION PROCESSING DEVICE, PRESENTATION METHOD, AND SURGICAL SYSTEM

(71) Applicant: SONY GROUP CORPORATION, TOKYO (JP)

(72) Inventors: KUNIHIKO AKIYOSHI, TOKYO (JP); JUN OKAMURA, TOKYO (JP); MASASHI NAITO, TOKYO (JP); HIROSHIGE HAYAKAWA, TOKYO (JP); AKIHIKO NAKATANI, TOKYO (JP)

(21) Appl. No.: 17/297,452

(22) PCT Filed: Nov. 25, 2019
(86) PCT No.: PCT/JP2019/045958

§ 371 Date: May 26, 2021

(30) Foreign Application Priority Data
Dec. 5, 2018 (JP) 2018-227826

(51) Int. Cl. A61B 90/90; A61B 90/00

(52) U.S. Cl. CPC A61B 90/90; A61B 2560/0493; A61B 2090/0803; A61B 90/08

(57) ABSTRACT

The present disclosure relates to an information processing device, a presentation method, and a surgical system that make it possible to suppress the occurrence of medical accidents. A voice recognition unit counts, through voice recognition, the remaining number of surgical tools existing in the body of the patient and used for a surgical operation, an image recognition unit counts, through image recognition, the remaining number of the surgical tools existing in the body of the patient, and a presentation control unit presents a predetermined warning when a difference arises between a first remaining number counted through the voice recognition and a second remaining number counted through the image recognition. The present disclosure can be applied to surgical systems.

### TECHNICAL FIELD

The present disclosure relates to an information processing device, a presentation method, and a surgical system, and more particularly, to an information processing device, a presentation method, and a surgical system that make it possible to suppress the occurrence of medical accidents.

### BACKGROUND ART

In surgical situations, it is required to suppress the occurrence of medical accidents in which the surgical operation is finished while an item used for the surgical operation, such as gauze or a surgical needle, is left in the patient's body.

Medical professionals including nurses visually check the number of items to be used for the surgical operation, and then count the number by vocalization or make sure that the number of remaining items is correct after the surgical operation so as to prevent the above-mentioned medical accidents. However, there is a non-negligible possibility that the above-mentioned medical accidents occur due to a human error.

Thus, for example, Patent Document 1 discloses a system for counting the number of pieces of gauze each having an IC tag attached, as a configuration for accurately counting the number of items used for a surgical operation.

### CITATION LIST

### Patent Document

Patent Document 1: Japanese Patent Application Laid-Open No. 2013-97761

### SUMMARY OF THE INVENTION

### Problems to be Solved by the Invention

However, the configuration described in Patent Document 1 is not enough to accurately count the number of items used for a surgical operation because the configuration cannot be applied to an item that is too small for an IC tag to be attached thereto, such as a surgical needle.

The present disclosure has been made in view of such circumstances, and is intended to suppress the occurrence of medical accidents.

### Solutions to Problems

An information processing device of the present disclosure includes: a voice recognition unit that counts, through voice recognition, a remaining number of surgical tools existing in a body of a patient and used for a surgical operation; an image recognition unit that counts, through image recognition, the remaining number of the surgical tools existing in the body of the patient; and a presentation control unit that presents a predetermined warning when a difference arises between a first remaining number counted through the voice recognition and a second remaining number counted through the image recognition.

A presentation method of the present disclosure includes: counting, through voice recognition, a remaining number of surgical tools existing in a body of a patient and used for a surgical operation, the counting being performed by an information processing device; counting, through image recognition, the remaining number of the surgical tools existing in the body of the patient, the counting being performed by the information processing device; and presenting a predetermined warning when a difference arises between a first remaining number counted through the voice recognition and a second remaining number counted through the image recognition, the presenting being performed by the information processing device.

A surgical system of the present disclosure includes: a voice recognition unit that counts, through voice recognition, a remaining number of surgical tools existing in a body of a patient and used for a surgical operation; an image recognition unit that counts, through image recognition, the remaining number of the surgical tools existing in the body of the patient; and a presentation control unit that presents a predetermined warning when a difference arises between a first remaining number counted through the voice recognition and a second remaining number counted through the image recognition.

In the present disclosure, the remaining number of surgical tools existing in a body of a patient and used for a surgical operation is counted through voice recognition, the remaining number of the surgical tools existing in the body of the patient is counted through image recognition, and a predetermined warning is presented when a difference arises between a first remaining number counted through the voice recognition and a second remaining number counted through the image recognition.

### BRIEF DESCRIPTION OF DRAWINGS

FIG. 1 is a schematic diagram illustrating an example configuration of a surgical system according to the present embodiment.

FIG. 2 is a block diagram illustrating an example configuration of the surgical system.

FIG. 3 is a block diagram illustrating an example functional configuration of an operating room server.

FIG. 4 is a flowchart explaining a flow of a warning presentation process.

FIG. 5 is a diagram illustrating an example screen displayed on a monitor.

FIG. 6 is a diagram illustrating an example screen displayed on a monitor.

FIG. 7 is a diagram explaining image capturing in the case of inconsistent counted numbers.

FIG. 8 is a diagram illustrating an example screen displayed on a monitor.

FIG. 9 is a diagram illustrating an example screen displayed on a monitor.

FIG. 10 is a schematic diagram illustrating another example configuration of the surgical system.

FIG. 11 is a flowchart explaining a flow of a warning presentation process.

FIG. 12 is a block diagram illustrating an example hardware configuration of the operating room server.

A mode for carrying out the present disclosure (hereinafter referred to as an embodiment) will now be described. Note that descriptions will be provided in the order mentioned below.

1. System configuration

2. Configuration and operation of operating room server

3. Examples of displayed screen

4. Timing to calculate difference between remaining numbers

5. Modifications

6. Use cases

7. Hardware configuration

<1. System Configuration>

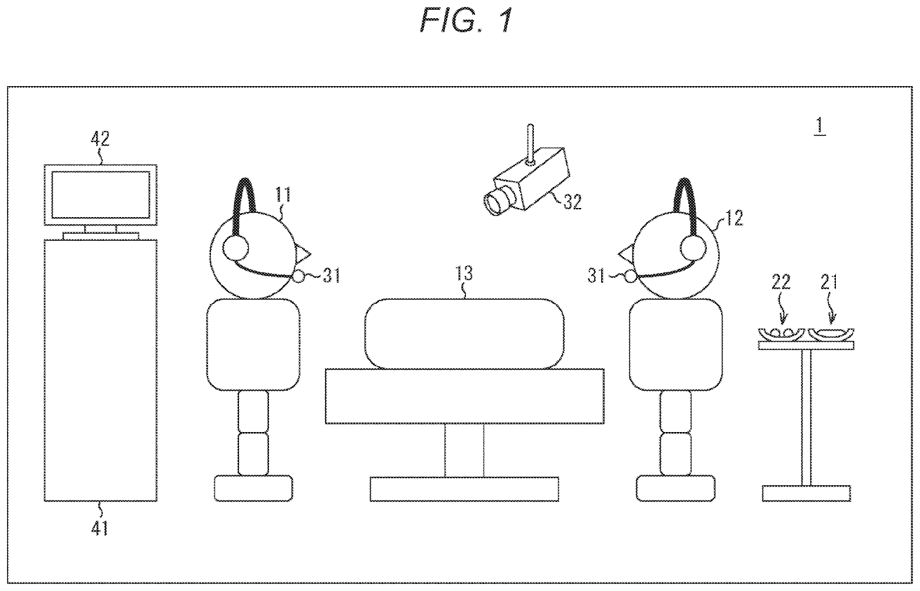

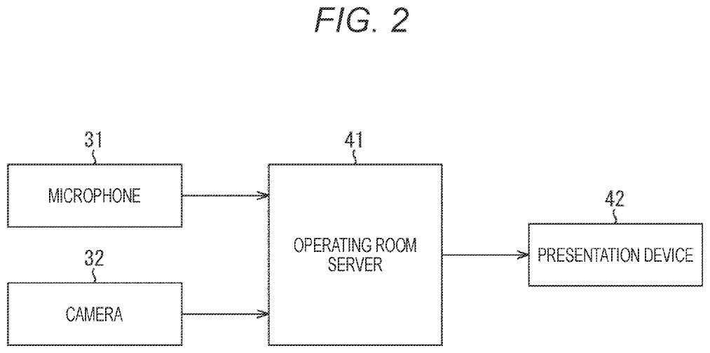

FIG. 1 is a schematic diagram illustrating an example configuration of a surgical system according to the present embodiment, and FIG. 2 is a block diagram illustrating an example configuration of the surgical system.

FIG. 1 shows that, in an operating room including the surgical system 1, a surgeon 11 and a scrub nurse 12 (a nurse preparing surgical instruments) are standing while facing each other across a patient 13 on a surgical table.

On the table behind the nurse 12, surgical tools used for the surgical operation are placed including a hygiene material 21 such as gauze and a surgical instrument 22 such as a surgical needle. The hygiene material 21 and the surgical instrument 22 are handed to the surgeon 11 by the nurse 12. The hygiene material 21 includes, for example, a pledget, a sponge, an anti-adhesion material, and the like as well as gauze, and the surgical instrument 22 includes, for example, a scalpel, scissors, forceps, tweezers, and the like as well as a surgical needle.

The surgeon 11 and the nurse 12 each wear a headset-type microphone 31. In addition, a camera 32 is installed on the ceiling of the operating room so as to see the patient 13 and surroundings of the patient 13 from above. In the example in FIG. 1, only one camera 32 is installed, but a plurality of cameras 32 may be installed.

Behind the surgeon 11, an operating room server 41 and a presentation device 42 are installed. The presentation device 42 is configured as a monitor and/or a speaker to present information to the surgeon 11, the nurse 12, and other operators in the operating room on a display and/or by outputting a sound under the control of the operating room server 41. The operating room server 41 may be installed outside the operating room.

In surgical situations, it is conventionally required to suppress the occurrence of medical accidents in which the surgical operation is finished while a surgical tool such as gauze or a surgical needle is left in the patient's body. For this purpose, for example, either one of the following needs to be assured (on condition that gauze is not broken):

(1) the number of pieces of the gauze existing in the patient's body is 0; and

(2) the number of pieces of the gauze inserted into the patient's body is equal to the number of pieces of the gauze removed from the patient's body.

A conceivable solution to achieve either one of the two above is, for example, counting the number of pieces of the gauze on the basis of an image taken by the camera 32.

However, when the gauze inserted into the patient's body is hidden by the hand of the surgeon 11 or moved inside the patient's body, it is difficult to count the number of pieces of the gauze, and thus (1) mentioned above is difficult to achieve. In addition, if a plurality of pieces of the gauze is inserted into the patient's body, it is difficult to recognize the number of the pieces, and thus (2) mentioned above is also difficult to achieve.

Another conceivable solution to achieve either one of the two above is counting the number of pieces of the gauze on the basis of a voice given by the surgeon 11 or the nurse 12.

However, (1) above is difficult to achieve unless there is no wrong counting or no wrong utterance of the number of pieces of gauze. In addition, (2) is also difficult to achieve unless the number of a plurality of pieces of the gauze is uttered when the pieces of the gauze are taken out of the patient's body.

Therefore, in the surgical system 1 according to the present embodiment, the operating room server 41 counts the number of surgical tools in the patient's body on the basis of a voice given by the surgeon 11 or the nurse 12 as input from the microphone 31 and an image taken by the camera 32.

Furthermore, when a difference arises between the number counted on the basis of a voice and the number counted on the basis of an image, the operating room server 41 causes the presentation device 42 to present a predetermined warning.

As a result, it can be ensured that the occurrence of medical accidents in which the surgical operation is finished while a surgical tool such as gauze or a surgical needle is left in the patient's body is suppressed.

<2. Configuration and Operation of Operating Room Server>

Now, the following describes a configuration and operation of the operating room server 41, which achieves suppression of the occurrence of medical accidents as described above.

(Configuration of Operating Room Server)

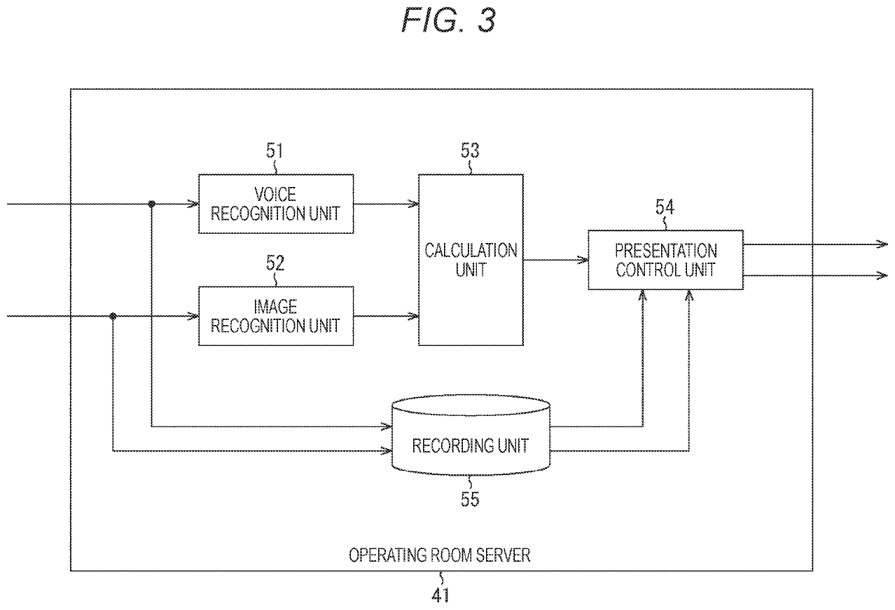

FIG. 3 is a block diagram illustrating an example functional configuration of the operating room server 41 serving as an information processing device according to an embodiment of the present disclosure.

The operating room server 41 in FIG. 3 includes a voice recognition unit 51, an image recognition unit 52, a calculation unit 53, a presentation control unit 54, and a recording unit 55.

The voice recognition unit 51 counts the remaining number of surgical tools existing in the patient's body through voice recognition on the basis of utterances given by the surgeon 11 and the nurse 12 (mainly the nurse 12) as input from the microphone 31. The remaining number of surgical tools counted through voice recognition (hereinafter also referred to as the voice count) is supplied to the calculation unit 53.

The image recognition unit 52 counts the remaining number of surgical tools existing in the patient's body through image recognition on the basis of an image taken by the camera 32. The remaining number of surgical tools counted through image recognition (hereinafter also referred to as the image count) is supplied to the calculation unit 53.

The calculation unit 53 calculates the difference between the voice count supplied from the voice recognition unit 51 and the image count supplied from the image recognition unit 52. The information representing the voice count, the image count, and the difference therebetween is supplied to the presentation control unit 54.

The presentation control unit 54 controls the presentation of information on the presentation device 42 by displaying information or outputting a sound. The presentation control unit 54 presents the voice count and the image count on the basis of the information from the calculation unit 53 and, when a difference arises between the voice count and the image count, presents a predetermined warning.

The recording unit 55 records sounds input from the microphone 31 and images taken by the camera 32 during a surgical operation. If necessary, any of the recorded sounds and images is presented to the presentation device 42 by the presentation control unit 54.

(Operation of Operating Room Server)

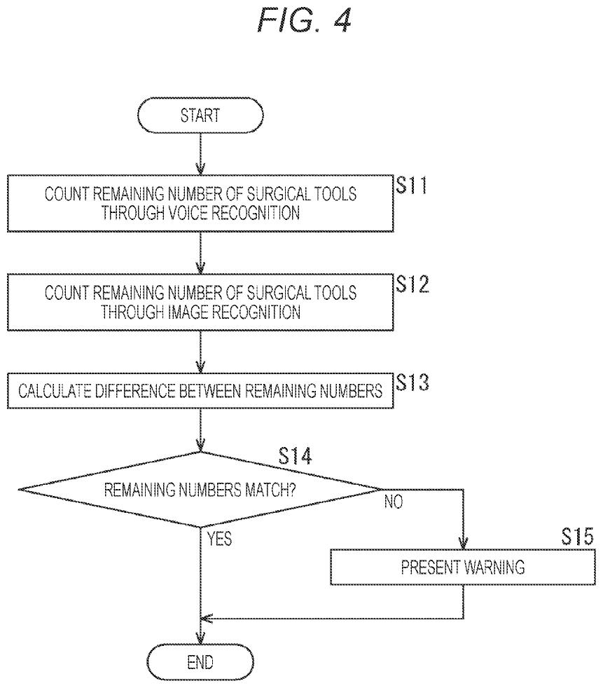

Next, referring to the flowchart in FIG. 4, the following describes a flow of a warning presentation process carried out by the operating room server 41.

In step S11, the voice recognition unit 51 counts the remaining number of surgical tools existing in the body of the patient 13 through voice recognition.

For example, the voice recognition unit 51 counts the remaining number of surgical tools existing in the body of the patient 13 by using a difference between the number of insertions, which is the number of times a surgical tool is inserted into the body of the patient 13, and the number of removals, which is the number of times a surgical tool is removed from the body of the patient 13, as counted through voice recognition.

In this step, the voice recognition unit 51 may count the remaining number of surgical tools existing in the body of the patient 13 through voice recognition on utterances given by a plurality of operators including the nurse 12 and any other nurses.

It is assumed that words to be voice-recognized are registered in advance. For example, words like “putting in” and “taking out” each representing the operation of insertion or removal, “gauze put in” and “surgical needle taken out” each representing the name of a surgical tool and the operation of insertion or removal, and the like are registered in advance. Furthermore, the number of surgical tools may be voice-recognized, such as “three pieces of gauze put in”.

In step S12, the image recognition unit 52 counts the remaining number of surgical tools existing in the body of the patient 13 through image recognition.

For example, the image recognition unit 52 counts the remaining number of surgical tools existing in the body of the patient 13 by using a difference between the number of insertions, which is the number of times a surgical tool is inserted into the body of the patient 13, and the number of removals, which is the number of times a surgical tool is removed from the body of the patient 13, as counted through image recognition.

In this step, the image recognition unit 52 may count the remaining number of surgical tools existing in the body of the patient 13 through image recognition on a plurality of moving images showing a surgical tool captured by a plurality of cameras 32.

The image-recognized surgical tool is an object learned by machine learning. In this step, objects that are usually unlikely to be left in the patient's body, such as elongated items like a stent, forceps, and the like, may be excluded from the objects to be recognized.

For example, a learning model having a predetermined parameter is generated by inputting, to a multi-layer neural network, learning data in which a captured image showing a surgical tool is associated with the surgical tool appearing in the image. Then, an image taken by the camera 32 is input to the generated learning model, so that it is determined whether or not a surgical tool is shown in the image. Note that such machine learning is only required to make it possible to determine whether or not a surgical tool is present, and reinforcement learning, for example, may be applied to the machine learning. Furthermore, an area showing a surgical tool may be identified by using, as learning data, an image to which an annotation indicating the area showing a surgical tool is added.

Moreover, for voice recognition, a learning model may also be generated by inputting, to a multi-layer neural network, learning data in which a sound produced during the surgical operation including an utterance given by a person as recorded by using a sound input device such as a microphone is associated with an annotation indicating a point of utterance given by a person. In this case, the accuracy of voice recognition can be improved by performing sound separation on the sound that has been input from the sound input device to separate an utterance sound of a person from the sound and performing voice recognition on the separated utterance sound. Note that, for voice recognition, a learning model for voice recognition may also be generated and used by doing machine learning based on learning data in which a voice is associated with a process corresponding to a voice.

Furthermore, for example, if a plurality of pieces of gauze is inserted into the body of the patient 13 and removed from the body of the patient 13, and then no gauze is image-recognized in the body of the patient 13, the number may be counted on the assumption that all the plurality of inserted pieces of gauze has been removed from the body of the patient 13.

Moreover, for example, although the gauze containing absorbed blood and a surface of an organ are in a similar red color, a piece of the gauze and the organ may be separately recognized on the basis of the contrast in color information.

The process of step S11 is performed every time the number of insertions or the number of removals is counted through voice recognition. In addition, the process of step S12 is performed every time the number of insertions or the number of removals is counted through image recognition.

At a predetermined timing, in step S13, the calculation unit 53 calculates the difference between the remaining number of surgical tools counted through voice recognition and the remaining number of surgical tools counted through image recognition.

In step S14, the calculation unit 53 determines whether or not the remaining numbers (the voice count and the image count) match.

If the voice count and the image count match, the presentation control unit 54 does nothing and the processing is ended.

On the other hand, if the voice count and the image count do not match, the processing goes to step S15, and the presentation control unit 54 causes the presentation device 42 to present a warning.

According to the above-described processing, a warning will be presented on the presentation device 42 when the voice count and the image count do not match, that is, when either the voice count or the image count is wrong. Therefore, the nurse 12 preparing surgical instruments and a circulating nurse (not illustrated) have opportunities to count the number of surgical tools placed on the table and to ask the surgeon 11 to check the number of surgical tools existing in the body of the patient 13. As a result, it is made possible to suppress the occurrence of a medical accident in which the surgical operation is finished while a surgical tool is left in the patient's body.

<3. Examples of Displayed Screen>

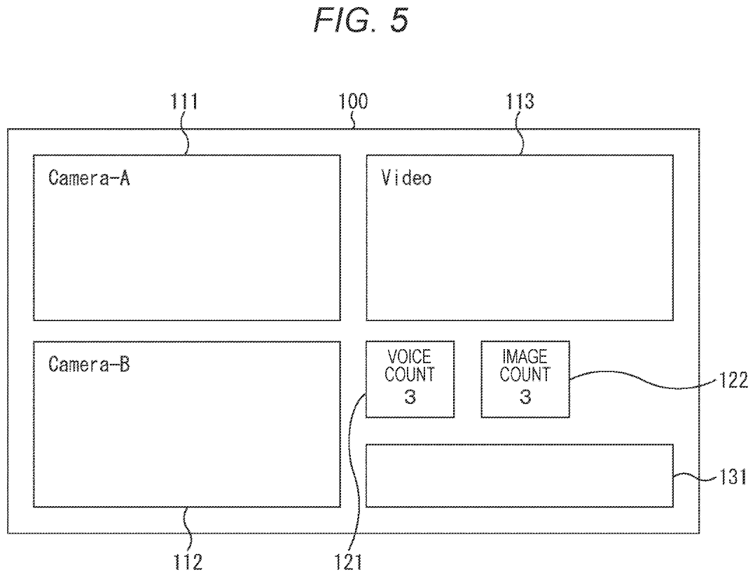

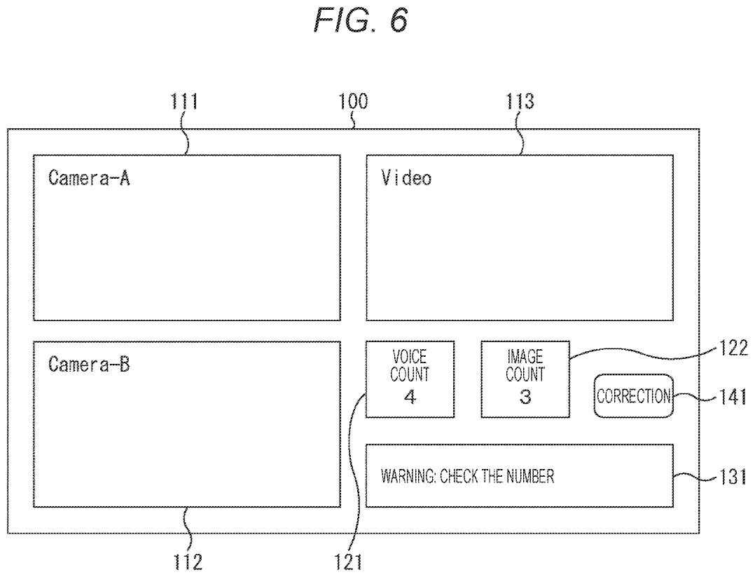

Now, referring to FIGS. 5 and 6, the following describes examples of a screen displayed on the presentation device 42 configured as a monitor.

On the left half of a screen 100 illustrated in FIGS. 5 and 6, there are vertically arranged display areas 111 and 112 showing operative field images in real time as provided by the two cameras 32 taking images of the operative field.

Furthermore, on the upper side of the right half of the screen 100, there is provided a display area 113 showing vital signs and other information, images of the operative field previously recorded, and the like.

On the screen 100, a voice count display part 121 and an image count display part 122 are provided under the display area 113, and a warning display part 131 is provided under these count display parts.

The voice count display part 121 shows the remaining number of surgical tools (voice count) existing in the body of the patient 13, as counted through voice recognition. In addition, the image count display part 122 shows the remaining number of surgical tools (image count) existing in the body of the patient 13, as counted through image recognition.

In this example, the remaining number of all surgical tools is counted and displayed without regard to types of surgical tools (regardless of whether the surgical tool is the hygiene material 21 such as gauze or the surgical instrument 22 such as a surgical needle).

In the example in FIG. 5, the voice count and the image count match as both are “3”, and nothing is displayed in the warning display part 131.

In contrast, the example in FIG. 6 shows that the counts do not match as the voice count is “4” while the image count is “3”. In such cases, a warning message like “Warning: Check the number” is displayed in the warning display part 131. At the same time, the presentation device 42 may output a sound of a message similar to the warning message.

Having seen (heard) the warning message, the nurse 12 preparing surgical instruments and a circulating nurse have opportunities to count the number of surgical tools placed on the table and to ask the surgeon 11 to check the number of surgical tools existing in the body of the patient 13.

Note that the example in FIG. 6 shows, to the right of the image count display part 122, a correction button 141 for accepting correction of the voice count and the image count. If any of the displayed counts is wrong, as confirmed by the circulating nurse after asking the surgeon 11 to check the number of surgical tools existing in the body of the patient 13 or checking by the circulating nurse him/herself the number of surgical tools in use and the number of discarded surgical tools, the voice count or the image count can be corrected by pressing the correction button 141.

Furthermore, as described above, sounds input from the microphone 31 and images taken by the camera 32 during the surgical operation are recorded in the recording unit 55. Therefore, a voice or an image as of the time when the voice count and the image count do not match (a voice or an image that may be a cause of the discrepancy) may be presented.

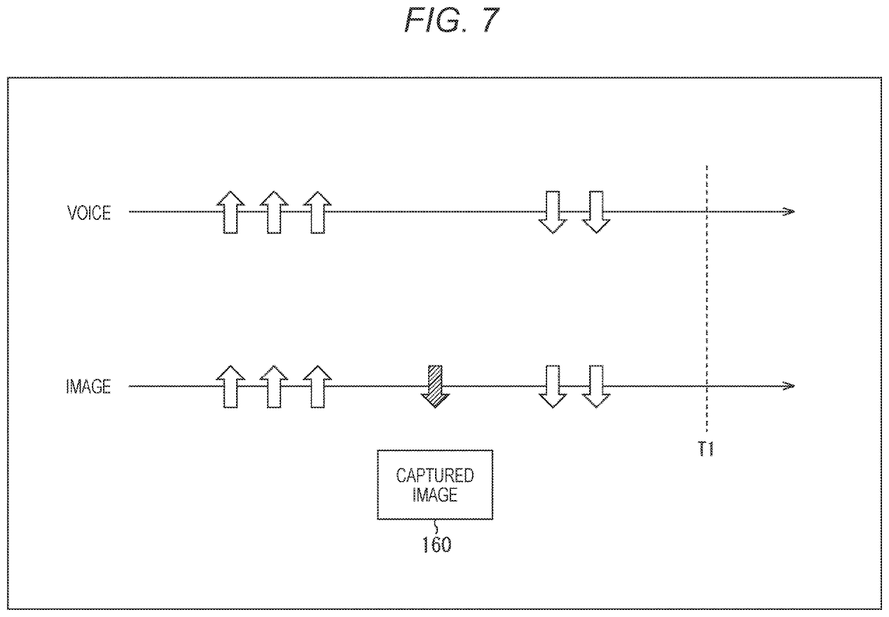

FIG. 7 is a diagram explaining image capturing performed when the voice count and the image count do not match.

The upper part of FIG. 7 shows the voice count along the time axis while the lower part of FIG. 7 shows the image count along the time axis. In the figure, an up arrow on the time axis indicates that a surgical tool has been inserted into the body of the patient 13, and a down arrow on the time axis indicates that a surgical tool has been removed from the body of the patient 13.

According to the voice count as of time T1, the number of insertions is 3 and the number of removals is 2, and thus the remaining number is 1. On the other hand, according to the image count, the number of insertions is 3 and the number of removals is 3, and thus the remaining number is 0. That is, the voice count and the image count do not match.

In such cases, on the basis of the sounds and images recorded in the recording unit 55, the presentation control unit 54 infers a timing at which the voice count and the image count become inconsistent, and extracts the voice and image as of the timing.

In the example in FIG. 7, a still image as of the timing of the first removal (the hatched down arrow) in the image count is captured as a captured image 160. The captured image 160 is displayed in the display area 113 on the screen 100, so that the nurse 12 preparing surgical instruments or the circulating nurse can check the situation as of the timing.

In particular, if the number of insertions is different between the voice count and the image count, there is a possibility that an extra surgical tool remains in the body of the patient 13. In this case, the nurse 12 preparing surgical instruments or the circulating nurse can check the captured image, and then ask the surgeon 11 to check the number of surgical tools existing in the body of the patient 13.

In addition, the examples in FIGS. 5 and 6 show that merely the remaining numbers of surgical tools as counted through voice recognition and image recognition are displayed.

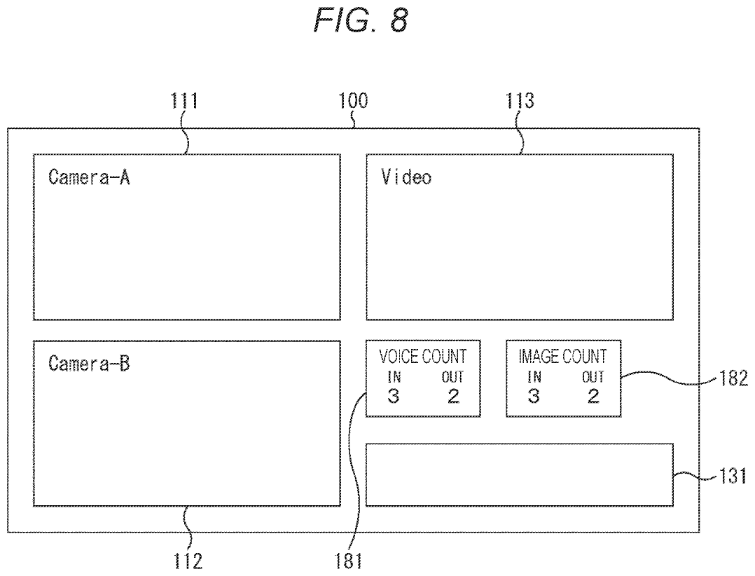

However, this is not restrictive; as illustrated in FIG. 8, the number of insertions and the number of removals counted through voice recognition and the number of insertions and the number of removals counted through image recognition may be displayed.

In the example in FIG. 8, a voice count display part 181 and an image count display part 182 are provided on the screen 100 instead of the voice count display part 121 and the image count display part 122.

The voice count display part 181 displays the number of insertions (IN) and the number of removals (OUT) counted through voice recognition. In addition, the image count display part 182 displays the number of insertions (IN) and the number of removals (OUT) counted through image recognition.

In the example in FIG. 8, the voice count and the image count for the number of insertions match as both are “3” and the voice count and the image count for the number of removals match as both are “2”, and nothing is displayed in the warning display part 131.

In addition, in the above-described examples, the remaining number of all surgical tools is counted and displayed without regard to types of surgical tools.

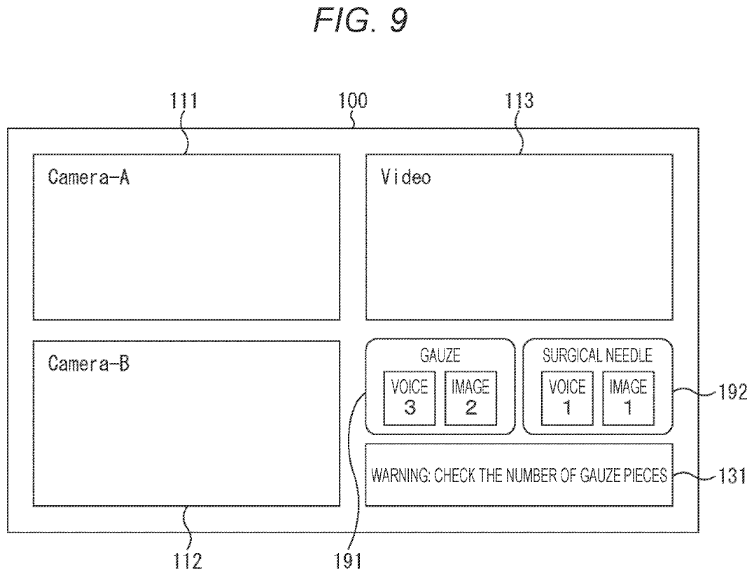

However, this is not restrictive; as illustrated in FIG. 9, the remaining number of surgical tools may be counted and displayed for each type of surgical tools.

In the example in FIG. 9, a gauze count display part 191 and a surgical needle count display part 192 are provided on the screen 100 instead of the voice count display part 121 and the image count display part 122.

The gauze count display part 191 displays the gauze voice count counted through voice recognition and the gauze image count counted through image recognition. Furthermore, the surgical needle count display part 192 displays the surgical needle voice count counted through voice recognition and the surgical needle image count counted through image recognition.

In the example in FIG. 9, while the surgical needle count display part 192 shows consistent numbers as both the voice count and the image count is “1”, the gauze count display part 191 shows inconsistent numbers as the voice count is “3” and the image count is “2”. In such cases, a warning message like “Warning: Check the number of gauze pieces” is displayed in the warning display part 131. At the same time, the presentation device 42 may output a sound of a message similar to the warning message.

Moreover, although not illustrated, a combination of the example in FIG. 8 and the example in FIG. 9 may be displayed; that is, the number of insertions and the number of removals counted through voice recognition and the number of insertions and the number of removals counted through image recognition may be displayed for each type of surgical tools.

Furthermore, the correction button 141 in FIG. 6 may be provided on the screen 100 in any of the example in FIG. 8, the example in FIG. 9, and the example of a combination of the example in FIG. 8 and the example in FIG. 9.

<4. Timing to Calculate Difference Between Remaining Numbers>

The following describes examples of the timing (the timing to present a warning) when the calculation unit 53 calculates the difference between the remaining number of surgical tools counted through voice recognition (voice count) and the remaining number of surgical tools counted through image recognition (image count).

(Timing when the Number is Counted Through Voice Recognition)

Typically, after handing a surgical tool to the surgeon 11, the nurse 12 utters the name or the like of the surgical tool. Therefore, for example, when the voice recognition unit 51 counts the remaining number of surgical tools, the difference between the voice count and the image count is calculated. Alternatively, the difference between the voice count and the image count may be calculated when a predetermined time (20 seconds, for example) has passed after the remaining number of surgical tools is counted by the voice recognition unit 51.

(Timing when the Number is Counted Through Image Recognition)

Contrary to the above-described example, when the image recognition unit 52 counts the remaining number of surgical tools, the difference between the voice count and the image count may be calculated. For example, at a time after the image recognition unit 52 counts the remaining number of surgical tools, such as, for example, 5 minutes later, the difference between the voice count and the image count is calculated.

In this case, unless the voice recognition unit 51 counts the remaining number of surgical tools within 5 minutes after the image recognition unit 52 counts the remaining number of surgical tools, a difference will arise between the voice count and the image count. Furthermore, if the voice recognition unit 51 starts counting the remaining number of surgical tools at a time, for example, 4 minutes and 59 seconds after the image recognition unit 52 counts the remaining number of surgical tools, the difference between the voice count and the image count is further calculated after one minute from that time point.

(Timing when Scenes are Switched)

At transition to a next operation step, images of the operative field taken by, for example, the camera 32 significantly change in background. Therefore, the difference between the voice count and the image count may be calculated when scenes in the operative field images are switched.

(Timing Dependent on Signal from Medical Device)

The difference between the voice count and the image count may be calculated in response to a signal from a medical device in use for the surgical operation. For example, the difference between the voice count and the image count is calculated when a signal is supplied from the electric scalpel in use for the surgical operation, the signal indicating that the electric scalpel has been energized.

(Timing at Regular Time Intervals)

The difference between the voice count and the image count may be calculated at regular time intervals such as, for example, every 10 minutes.

(Timing when Surgical Operation is Finished)

Eventually, the remaining number of surgical tools existing in the patient's body is only needed to be 0 when the surgical operation is finished. Therefore, the difference between the voice count and the image count may be calculated when the surgical operation is finished. A timing when the surgical operation is finished is the time when the surgical site is closed, such as the time when the abdominal suture is started in the case of an abdominal operation or the time when a predetermined time has passed after the scope is removed in the case of an endoscopic operation.

<5. Modifications>

(Configuration of Operating Room Server)

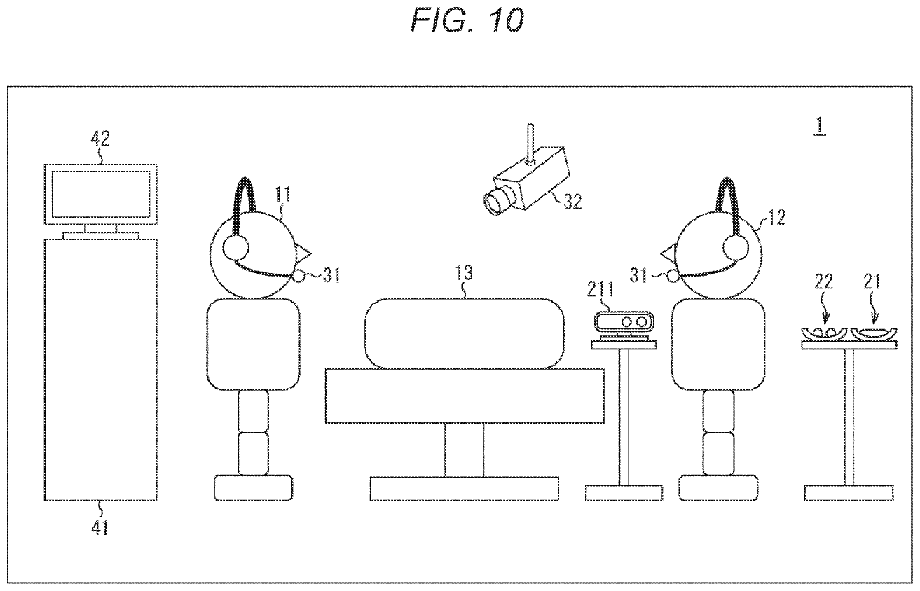

FIG. 10 is a schematic diagram illustrating another example configuration of the surgical system 1.

The surgical system 1 in FIG. 10 differs from the surgical system 1 in FIG. 1 in that an object passage sensor 211 is additionally provided.

The object passage sensor 211 includes, for example, a time-of-flight (ToF) camera or an infrared camera, and detects the passage of a surgical tool between the nurse 12 and the patient 13 or between the nurse 12 and the surgeon 11.

Note that the object passage sensor 211 may be configured as a camera for taking images of the hygiene material 21 and the surgical instrument 22, or may be configured as a polarization camera. In a case where the object passage sensor 211 is configured as a polarization camera, the hygiene material 21 being translucent and the surgical instrument 22 being in a silver color (including a metal) can be detected with high precision.

Although not illustrated, in the operating room server 41 in the surgical system 1 in FIG. 10, there is provided an object passage recognition unit that counts the remaining number of surgical tools existing in the patient's body on the basis of the number of objects that have been detected passing by the object passage sensor 211.

(Operation of Operating Room Server)

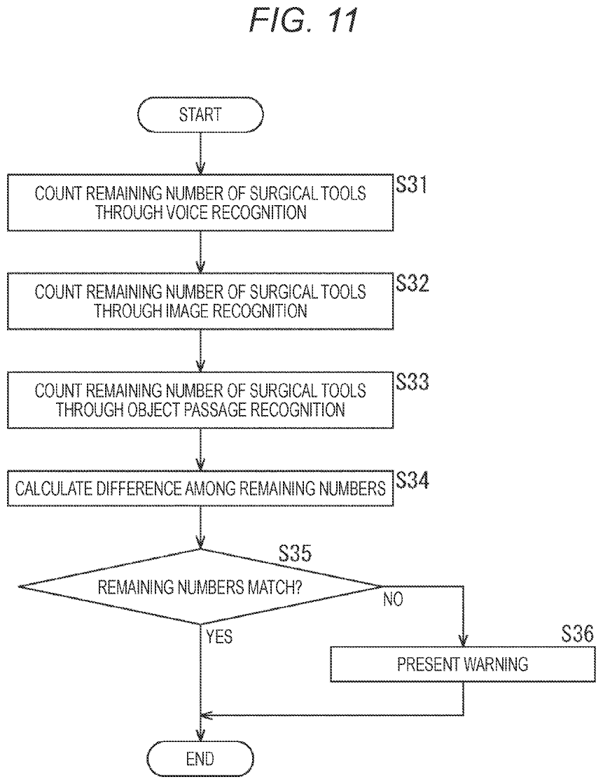

Next, referring to the flowchart in FIG. 11, the following describes a flow of a warning presentation process carried out by the operating room server 41 in FIG. 10.

Note that the processes in S31 and S32 in the flowchart in FIG. 11 are similar to the processes in S11 and S12 in the flowchart in FIG. 4, and thus the description thereof will be omitted.

In step S33 subsequent to step S32, the object passage recognition unit (not illustrated) counts the remaining number of surgical tools existing in the body of the patient 13 through object passage recognition.

In step S34, the calculation unit 53 calculates the individual differences among the remaining number of surgical tools counted through voice recognition, the remaining number of surgical tools counted through image recognition, and the remaining number of surgical tools counted through object passage recognition (hereinafter referred to as the object passage count).

In step S35, the calculation unit 53 determines whether or not the remaining numbers (the voice count, the image count, and the object passage count) match one another.

If all the voice count, the image count, and the object passage count match one another, the presentation control unit 54 does nothing and the processing is ended.

On the other hand, if there is any inconsistency among the voice count, the image count, and the object passage count, the processing goes to step S36, and the presentation control unit 54 causes the presentation device 42 to present a warning.

According to the above-described processing, a warning is presented on the presentation device 42 if there is any inconsistency among the voice count, the image count, and the object passage count, whereby it is made possible to suppress the occurrence of a medical accident in which the surgical operation is finished while a surgical tool is left in the patient's body.

<6. Use Cases>

The technology according to the present disclosure is applied to surgical systems for conducting various surgical operations.

For example, the technology according to the present disclosure can be applied to a surgical system for conducting surgical operations of brain tumors such as meningioma.

A surgical operation of a brain tumor mainly includes craniotomy, extirpation, and suture carried out in the order mentioned.

To deal with bleeding caused when the bone of skull is trephined, a plurality of pieces of gauze is inserted into the skull or removed therefrom. Furthermore, to extirpate the tumor existing in the dura, a surgical needle and a piece of gauze are inserted into the skull or removed therefrom. After the tumor is extirpated, the skin is sutured with, for example, an artificial dura applied, and the surgical operation is finished.

By applying the technology according to the present disclosure to such a surgical system for conducting surgical operations of brain tumors, it is made possible to suppress the occurrence of a medical accident in which the surgical operation is finished while a surgical needle or gauze is left in the skull of the patient.

Furthermore, the technology according to the present disclosure may be applied to a surgical system for conducting endoscopic operations.

In an endoscopic operation, a plurality of pieces of gauze is also inserted into the abdominal cavity or removed therefrom in order to, for example, protect other organs.

By applying the technology according to the present disclosure to a surgical system for conducting endoscopic operations, it is made possible to suppress the occurrence of a medical accident in which the surgical operation is finished while gauze is left in the abdominal cavity of the patient.

<7. Hardware Configuration>

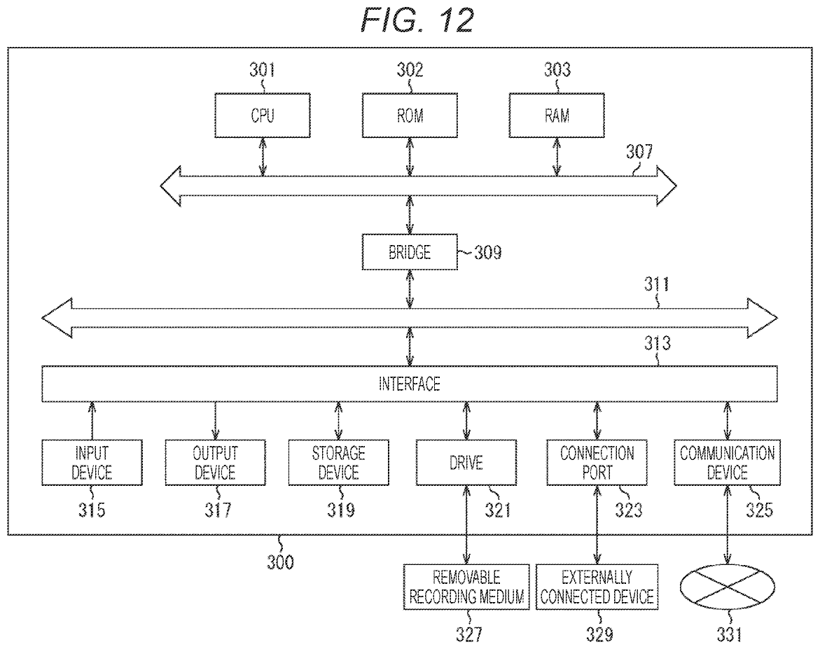

Next, referring to FIG. 12, the following describes in detail an example of a hardware configuration of the operating room server included in the surgical system according to the present embodiment.

FIG. 12 is a block diagram illustrating an example of the hardware configuration of the operating room server 300 included in the surgical system according to the present embodiment.

As shown in FIG. 12, the operating room server 300 includes a CPU 301, a ROM 303, and a RAM 305. Furthermore, the operating room server 300 includes a host bus 307, a bridge 309, an external bus 311, an interface 313, an input device 315, an output device 317, and a storage device 319. Note that the operating room server 300 may include a drive 321, a connection port 323, and a communication device 325.

The CPU 301 functions as an arithmetic processing device and a control device, and controls operations in the operating room server 300 in whole or in part in accordance with various programs recorded in the ROM 303, the RAM 305, the storage device 319, or a removable recording medium 327.

The ROM 303 stores programs, operation parameters, and the like to be used by the CPU 301. The RAM 305 primarily stores programs to be used by the CPU 301, parameters that vary as appropriate during execution of a program, and the like. These are connected to one another by the host bus 307 including an internal bus such as a CPU bus. Note that each of the components of the operating room server 41 as described with reference to FIG. 3 is implemented by, for example, the CPU 301.

The host bus 307 is connected to the external bus 311 such as a peripheral component interconnect/interface (PCI) bus via the bridge 309. To the external bus 311, the input device 315, the output device 317, the storage device 319, the drive 321, the connection port 323, and the communication device 325 are connected via the interface 313.

The input device 315 is operation means operated by the user, such as a mouse, a keyboard, a touch panel, a button, a switch, a lever, and a pedal, for example. Furthermore, the input device 315 may be, for example, remote control means (a so-called remote controller) employing infrared rays or other radio waves, or may be an externally connected device 329 supporting operation of the operating room server 300, such as a mobile phone and a PDA.

The input device 315 includes, for example, an input control circuit that generates an input signal on the basis of information input by the user by using the above-described operation means and outputs the generated input signal to the CPU 301.

By operating the input device 315, the user can input various types of data to the operating room server 300 and instruct the operating room server 300 to do processing operations.

The output device 317 includes a device that can visually or audibly give notification of the acquired information to the user. Specifically, the output device 317 is configured as a display device such as a CRT display device, a liquid crystal display device, a plasma display device, an EL display device, and a lamp, an audio output device such as a speaker and a headphone, a printer device, or the like.

The output device 317 outputs, for example, the results obtained by the operating room server 300 performing various types of processing. Specifically, the display device displays the results obtained by the operating room server 300 performing various types of processing in the form of text or images. On the other hand, the audio output device converts an audio signal including the reproduced audio data, acoustic data, and the like into an analog signal, and outputs the analog signal.

The storage device 319 is a data storage device configured as an example of the storage unit in the operating room server 300. The storage device 319 includes, for example, a magnetic storage unit device such as a hard disk drive (HDD), a semiconductor storage device, an optical storage device, or a magneto-optical storage device. The storage device 319 stores programs to be executed by the CPU 301, various types of data, and the like.

The drive 321 is a reader/writer for a recording medium, and is built in, or externally attached to, the operating room server 300. The drive 321 reads information recorded on the attached removable recording medium 327, such as a magnetic disk, an optical disk, a magneto-optical disk, or a semiconductor memory, and outputs the information to the RAM 305. Furthermore, the drive 321 is capable of writing a record onto the attached removable recording medium 327, such as a magnetic disk, an optical disk, a magneto-optical disk, or a semiconductor memory.

The removable recording medium 327 is, for example, a DVD medium, an HD-DVD medium, or a Blu-ray (registered trademark) medium. Furthermore, the removable recording medium 327 may be CompactFlash (registered trademark) (CF), a flash memory, a Secure Digital (SD) memory card, or the like. Moreover, the removable recording medium 327 may be, for example, an integrated circuit (IC) card on which a non-contact IC chip is mounted or an electronic device.

The connection port 323 is a port for directly connecting the externally connected device 329 to the operating room server 300. Examples of the connection port 323 include a universal serial bus (USB) port, an IEEE 1394 port, a small computer system interface (SCSI) port, and the like. Other examples of the connection port 323 include an RS-232C port, an optical audio terminal, a high-definition multimedia interface (HDMI (registered trademark)) port, and the like. By connecting the externally connected device 329 to the connection port 323, the operating room server 300 directly acquires various types of data from the externally connected device 329 and supplies various types of data to the externally connected device 329.

The communication device 325 is, for example, a communication interface including a communication device or the like for connecting to a communication network 331. The communication device 325 is, for example, a communication card or the like for a wired or wireless local area network (LAN), Bluetooth (registered trademark), or wireless USB (WUSB). Alternatively, the communication device 325 may be a router for optical communication, a router for asymmetric digital subscriber line (ADSL), a modem for various types of communication, or the like.

The communication device 325 is capable of transmitting and receiving signals to and from, for example, the Internet or another communication device in accordance with a predetermined protocol such as TCP/IP. Furthermore, the communication network 331 connected to the communication device 325 may be configured with a network or the like connected by wire or wirelessly. The communication network 331 may be, for example, the Internet or a home LAN, or may be a communication network on which infrared communication, radio wave communication, or satellite communication is carried out.

Each of the components of the above-described operating room server 300 may be configured by using a general-purpose member, or may be configured by using the hardware specialized for the functions of each of the components. Therefore, the hardware configuration to be used can be changed as appropriate in accordance with the technical level on an occasion of carrying out the present embodiment.

Moreover, it is possible to create a computer program for achieving the functions of the operating room server 300 included in the surgical system according to the present embodiment and implement the computer program on a personal computer or the like. Furthermore, it is also possible to provide a computer-readable recording medium containing such a computer program. The recording medium is, for example, a magnetic disk, an optical disk, a magneto-optical disk, a flash memory, or the like. Furthermore, the computer program may be distributed via, for example, a network without using a recording medium.

Embodiments of the present disclosure are not limited to the above-described embodiments, and various modifications can be made thereto without departing from the gist of the present disclosure.

For example, the present disclosure can be in a cloud computing configuration in which one function is distributed among, and handled in collaboration by, a plurality of devices via a network.

Furthermore, each of the steps described above with reference to the flowcharts can be executed not only by one device but also by a plurality of devices in a shared manner.

Moreover, in a case where one step includes a plurality of processes, the plurality of processes included in the one step can be executed not only by one device but also by a plurality of devices in a shared manner.

Furthermore, the present disclosure may have the following configurations.

(1)

An information processing device including:

a voice recognition unit that counts, through voice recognition, a remaining number of surgical tools existing in a body of a patient and used for a surgical operation;

an image recognition unit that counts, through image recognition, the remaining number of the surgical tools existing in the body of the patient; and

a presentation control unit that presents a predetermined warning when a difference arises between a first remaining number counted through the voice recognition and a second remaining number counted through the image recognition.

(2)

The information processing device according to (1), in which

the presentation control unit presents the first remaining number and the second remaining number.

(3)

The information processing device according to (1), in which

the voice recognition unit counts the first remaining number by using a difference between a first number of insertions, which is the number of times any of the surgical tools is inserted into the body of the patient, and a first number of removals, which is the number of times any of the surgical tools is removed from the body of the patient, the first number of insertions and the first number of removals being counted through the voice recognition, and

the image recognition unit counts the second remaining number by using a difference between a second number of insertions, which is the number of times any of the surgical tools is inserted into the body of the patient, and a second number of removals, which is the number of times any of the surgical tools is removed from the body of the patient, the second number of insertions and the second number of removals being counted through the image recognition.

(4)

The information processing device according to (3), in which

the presentation control unit presents the first number of insertions and the first number of removals, and the second number of insertions and the second number of removals.

(5)

The information processing device according to (1) or (2), further including:

a recording unit that records at least one of a voice or an image made during a surgical operation, in which

the presentation control unit presents at least one of the voice or the image made when a difference arises between the first remaining number and the second remaining number, on the basis of at least one of the voice or the image recorded in the recording unit.

(6)

The information processing device according to (1), (2), or (5), in which

the voice recognition unit counts the first remaining number for each type of the surgical tools,

the image recognition unit counts the second remaining number for each type of the surgical tools, and

the presentation control unit presents the first remaining number and the second remaining number for each type of the surgical tools.

(7)

The information processing device according to any of (1) to (6), in which

the presentation control unit accepts, after presenting the warning, correction of the first remaining number and the second remaining number.

(8)

The information processing device according to any of (1) to (7), in which

the voice recognition unit counts the first remaining number through the voice recognition on utterance given by one or more operators, and

the image recognition unit counts the second remaining number through the image recognition on one or more moving images in which any of the surgical tools is imaged.

(9)

The information processing device according to any of (1) to (8), further including:

a calculation unit that calculates, at a predetermined timing, a difference between the first remaining number and the second remaining number.

(10)

The information processing device according to (9), in which

the calculation unit calculates a difference between the first remaining number and the second remaining number when the first remaining number is counted by the voice recognition unit.

(11)

The information processing device according to (9), in which

the calculation unit calculates a difference between the first remaining number and the second remaining number when the second remaining number is counted by the image recognition unit.

(12)

The information processing device according to (9), in which

the calculation unit calculates a difference between the first remaining number and the second remaining number when scenes in an operative field image are switched.

(13)

The information processing device according to (9), in which

the calculation unit calculates a difference between the first remaining number and the second remaining number in response to a signal from a medical device used for the surgical operation.

(14)

The information processing device according to (13), in which

the signal is a signal indicating that an electric scalpel has been energized.

(15)

The information processing device according to (9), in which

the calculation unit calculates a difference between the first remaining number and the second remaining number at regular time intervals.

(16)

The information processing device according to (9), in which

the calculation unit calculates a difference between the first remaining number and the second remaining number when the surgical operation is finished.

(17)

The information processing device according to any of (1) to (16), in which

the surgical tools include at least one of a surgical instrument or a hygiene material.

(18)

The information processing device according to (17), in which

the surgical instrument includes a surgical needle, and

the hygiene material includes gauze.

(19)

A presentation method including:

counting, through voice recognition, a remaining number of surgical tools existing in a body of a patient and used for a surgical operation, the counting being performed by an information processing device;

counting, through image recognition, the remaining number of the surgical tools existing in the body of the patient, the counting being performed by the information processing device; and

presenting a predetermined warning when a difference arises between a first remaining number counted through the voice recognition and a second remaining number counted through the image recognition, the presenting being performed by the information processing device.

(20)

A surgical system including:

a voice recognition unit that counts, through voice recognition, a remaining number of surgical tools existing in a body of a patient and used for a surgical operation;

an image recognition unit that counts, through image recognition, the remaining number of the surgical tools existing in the body of the patient; and

a presentation control unit that presents a predetermined warning when a difference arises between a first remaining number counted through the voice recognition and a second remaining number counted through the image recognition.

### REFERENCE SIGNS LIST

1 Surgical system
31 Microphone
32 Camera
41 Operating room server
42 Presentation device
51 Voice recognition unit
52 Image recognition unit
53 Calculation unit
54 Presentation control unit
55 Recording unit

1. An information processing device comprising:
a voice recognition unit that counts, through voice recognition, a remaining number of surgical tools existing in a body of a patient and used for a surgical operation;
an image recognition unit that counts, through image recognition, the remaining number of the surgical tools existing in the body of the patient; and
a presentation control unit that presents a predetermined warning when a difference arises between a first remaining number counted through the voice recognition and a second remaining number counted through the image recognition.

2. The information processing device according to claim 1, wherein
the presentation control unit presents the first remaining number and the second remaining number.

3. The information processing device according to claim 1, wherein
the voice recognition unit counts the first remaining number by using a difference between a first number of insertions, which is the number of times any of the surgical tools is inserted into the body of the patient, and a first number of removals, which is the number of times any of the surgical tools is removed from the body of the patient, the first number of insertions and the first number of removals being counted through the voice recognition, and
the image recognition unit counts the second remaining number by using a difference between a second number of insertions, which is the number of times any of the surgical tools is inserted into the body of the patient, and a second number of removals, which is the number of times any of the surgical tools is removed from the body of the patient, the second number of insertions and the second number of removals being counted through the image recognition.

4. The information processing device according to claim 3, wherein
the presentation control unit presents the first number of insertions and the first number of removals, and the second number of insertions and the second number of removals.

5. The information processing device according to claim 1, further comprising:
a recording unit that records at least one of a voice or an image made during a surgical operation, wherein
the presentation control unit presents at least one of the voice or the image made when a difference arises between the first remaining number and the second remaining number, on a basis of at least one of the voice or the image recorded in the recording unit.

6. The information processing device according to claim 1, wherein
the voice recognition unit counts the first remaining number for each type of the surgical tools,
the image recognition unit counts the second remaining number for each type of the surgical tools, and
the presentation control unit presents the first remaining number and the second remaining number for each type of the surgical tools.

7. The information processing device according to claim 1, wherein
the presentation control unit accepts, after presenting the warning, correction of the first remaining number and the second remaining number.

8. The information processing device according to claim 1, wherein
the voice recognition unit counts the first remaining number through the voice recognition on utterance given by one or more operators, and
the image recognition unit counts the second remaining number through the image recognition on one or more moving images in which any of the surgical tools is imaged.

9. The information processing device according to claim 1, further comprising:
a calculation unit that calculates, at a predetermined timing, a difference between the first remaining number and the second remaining number.

10. The information processing device according to claim 9, wherein
the calculation unit calculates a difference between the first remaining number and the second remaining number when the first remaining number is counted by the voice recognition unit.

11. The information processing device according to claim 9, wherein
the calculation unit calculates a difference between the first remaining number and the second remaining number when the second remaining number is counted by the image recognition unit.

12. The information processing device according to claim 9, wherein
the calculation unit calculates a difference between the first remaining number and the second remaining number when scenes in an operative field image are switched.

13. The information processing device according to claim 9, wherein
the calculation unit calculates a difference between the first remaining number and the second remaining number in response to a signal from a medical device used for the surgical operation.

14. The information processing device according to claim 13, wherein
the signal is a signal indicating that an electric scalpel has been energized.

15. The information processing device according to claim 9, wherein
the calculation unit calculates a difference between the first remaining number and the second remaining number at regular time intervals.

16. The information processing device according to claim 9, wherein
the calculation unit calculates a difference between the first remaining number and the second remaining number when the surgical operation is finished.

17. The information processing device according to claim 1, wherein
the surgical tools include at least one of a surgical instrument or a hygiene material.

18. The information processing device according to claim 17, wherein
the surgical instrument includes a surgical needle, and
the hygiene material includes gauze.

19. A presentation method comprising:
counting, through voice recognition, a remaining number of surgical tools existing in a body of a patient and used for a surgical operation, the counting being performed by an information processing device;
counting, through image recognition, the remaining number of the surgical tools existing in the body of the patient, the counting being performed by the information processing device; and
presenting a predetermined warning when a difference arises between a first remaining number counted through the voice recognition and a second remaining number counted through the image recognition, the presenting being performed by the information processing device.

20. A surgical system comprising:
a voice recognition unit that counts, through voice recognition, a remaining number of surgical tools existing in a body of a patient and used for a surgical operation;
an image recognition unit that counts, through image recognition, the remaining number of the surgical tools existing in the body of the patient; and
a presentation control unit that presents a predetermined warning when a difference arises between a first remaining number counted through the voice recognition and a second remaining number counted through the image recognition.
